(19) United States
(12) Patent Application Publication
(10) Pub. No.: US 2022/0387328 A1
(43) Pub. Date: Dec. 8, 2022

(54) DOSAGE FORM FOR NICOTINE REPLACEMENT THERAPY

(71) Applicant: GlaxoSmithKline Consumer Healthcare Holdings (US) LLC, Wilmington, DE (US)

(72) Inventors: Jacqueline Ann Erickson, Warren, NJ (US); Muralikrishnan Angamuthu, Warren, NJ (US); Satish Kamath, Warren, NJ (US); Gary Fagan, Warren, NJ (US)

(21) Appl. No.: 17/338,747

(22) Filed: Jun. 4, 2021

(51) Int. Cl. A61K 9/20; A61K 9/00; A61K 31/465

(52) U.S. Cl. CPC A61K 9/2018; A61K 9/0056; A61K 9/2054; A61K 9/2027; A61K 31/465; A61K 9/2095

(57) ABSTRACT

A novel dosage form for intra-oral delivery of nicotine for use in nicotine replacement therapy; a process for preparation of said dosage form and intermediate compositions therefor; and methods for treatment.

### FIELD OF THE INVENTION

The present invention relates to a pharmaceutical dosage form for intra-oral delivery of nicotine; a process and intermediate compositions for the manufacture thereof; and methods for treatment. In particular, the invention relates to a novel tablet for use in nicotine replacement therapy.

### BACKGROUND OF THE INVENTION

The primary therapeutic use of nicotine is treating nicotine dependence to eliminate smoking and the damage it does to health. The objective is to supply low doses of nicotine to the subject while avoiding the toxins associated with smoking in order to aid in smoking cessation through the relief of nicotine withdrawal symptoms.

Several forms of nicotine replacement therapy (NRT) are available, including long-acting forms, such as the nicotine patch, as well as short-acting forms, such as the lozenge, chewing gum, nasal spray, and vapor inhaler.

The short-acting forms allow smokers to self-administer the amount of nicotine needed, in order to achieve a consistent concentration of nicotine in the blood, and to ward off acute nicotine cravings. The efficacy is similar as between all of these forms; but their pharmacokinetics (PKs) differ in terms of their maximal plasma nicotine levels (Cₘₐₓ), the time to reach Cₘₐₓ (Tₘₐₓ), and their half-lives (t_{1/2}) (see, e.g., Lindson N et al., “Different doses, durations and modes of delivery of nicotine replacement therapy for smoking cessation,” Cochrane Database Syst Rev. 2019; 4:CD013308).

The multiple formulations and non-prescription status of many NRT formats enable broad access and the ability of smokers to choose the format that fits their preference and lifestyle, which may improve their chance of successfully quitting smoking.

Of the oral forms, the hard lozenge products marketed over-the-counter in the U.S. under the Commit® or Nicorette® brands (classified by FDA as “troche/lozenges”), are often preferred to gums as they do not require chewing, and in some circumstances may be more socially acceptable. The 2 and 4 mg nicotine troche/lozenges were demonstrated to improve the rates of abstinence from smoking compared with placebo, with treatment effects lasting for at least 1 year (see, e.g., Shiffman S et al., “Efficacy of a nicotine lozenge for smoking cessation,” Arch Intern Med. 2002; 162:1267-76). The “mini”-troche/lozenge that is also marketed under the Nicorette® brand affords another dosage form option which delivers the same amount of nicotine as the larger variety, but with a faster dissolution rate in vitro (see, e.g., Nides M et al., “Nicotine lozenges in the relief of behaviorally provoked craving, Am J Health Behav. 2018; 42:69-79).

Troche/lozenges are solid oral preparations intended to dissolve or disintegrate slowly in the mouth; they are not meant to be chewed or swallowed, nor taken with liquids. They contain one or more medicaments, usually in a flavored, sweetened base. They can be prepared by molding (gelatin and/or fused sucrose or sorbitol base) or by compression of sugar-based tablets. Molded lozenges are sometimes referred to as “pastilles” while compressed lozenges are often referred to as “troches,” USP31-NF26. The term “lozenge” or “troche/lozenge” (the latter being employed herein for consistency with FDA usage), shall refer to a hard lozenge formed by compression.

The Commit®/Nicorette® 2 and 4 mg troche/lozenge and mini-troche/lozenge comprise nicotine active agent in the resin-bound form known as Nicotine Polacrilex, together with one or more buffering agents, as well as one or more other, inactive ingredients suitable for compression, e.g, bulking agents/fillers or diluents, dissolution modifiers and binding agents. Still other ingredients may include taste masking agents, flavors, sweeteners, chelating agents, antioxidants, preservatives, processing aids such as glidants or lubricants, and colorants, etc.

Manufacture of troche/lozenges has typically involved the intermediate step of preparing a “master granule” from powder excipients by solvent-based (i.e. “wet”) granulation and drying, followed by the blending of the master granules with the nicotine active and extra-granular excipients, then compression, optionally followed by application of a film or other coating. In an embodiment of the wet granulation process utilized for the Nicorette® mini-troche/lozenge, the buffering agent is divided between the master granule and the extra-granular portion, see, e.g., U.S. Pat. Nos. 8,501,164 and 8,940,772. In another embodiment, the nicotine has been included in the intra-granular portion, see, e.g., U.S. Published Application 2017/0172995.

In order to encourage continued compliance by subjects already maintaining a nicotine replacement regimen, as well as to attract new subjects in need of NRT, the inventors hereof have continually sought to devise new, enhanced NRT products having consumer appeal.

In particular, it has been an object to devise a therapeutic nicotine active agent-containing intra-oral dosage form that pairs the troche/lozenge format with a distinct sensory experience.

It has also been an object to devise such a dosage form so that it affords craving relief comparable to that associated with the Nicorette® marketed products.

It has been a particular objective to devise a novel tableted dosage form having distinct sensory features, which is also bioequivalent to the Nicorette® mini-troche/lozenge.

Desirably, such a dosage form would also be similar in dimensions to the Nicorette® mini-troche/lozenge.

Furthermore, it has been an important objective to develop a more economical process for manufacturing tableted dosage forms according to the invention.

### SUMMARY OF THE INVENTION

The inventors have devised a novel tablet for intra-oral delivery of nicotine to a subject in need thereof which comprises (a) an orally disintegrating portion comprising a sensory marker/signal, and (b) a troche/lozenge portion comprising a nicotine active agent.

The sensory marker/signal, which can be, for example, an organoleptic stimulus, and especially a taste-affecting stimulus, is intended to be perceived by the user as a “burst” of sensory stimulation that occurs nearly immediately upon administration as a result of the disintegration of this portion of the tablet in the oral cavity and release of the sensory/marker signal.

Advantageously, the inventors have found that the initial burst of sensory stimulus provides an organoleptic experience that can serve as a distinct signature of the product. In another aspect, it can serve as a “marker” or “signal” that telegraphs to the user that nicotine relief is on its way. In a further aspect, the burst of sensory stimulation can also usefully memorialize for the user each tablet administration so as to deter over-medication. Perhaps most importantly, it is anticipated that the novel organoleptic experience created by the tablet, which in a preferred aspect will comprise a flavor or flavor combination pleasing to the consumer, will encourage increased compliance by subjects already receiving NRT, as well as attract new subjects in need thereof.

During and following disintegration of the orally disintegrating portion, whereby the sensory marker/signal is released into the oral cavity within about a minute or less of administration, the nicotine active agent-containing portion, which shall be referred to as the “troche/lozenge portion,” is meant to be hold in the oral cavity for a period of time, e.g., from several minutes to at most about 30 minutes, and preferably from about 5 to about 20 minutes (e.g., from about 10 to about 15 minutes), during which time, optionally upon sucking or other oral manipulation by the user, this portion hydrates and disintegrates/dissolves, gradually releasing nicotine into the buccal tissue in a form suitable for uptake by the bloodstream.

It was not apparent prior to the present invention whether a tablet comprising an orally disintegrating portion in addition to a nicotine active agent-containing troche/lozenge portion, yet constrained to a size similar to the NICORETTE® mini-troche/lozenge, could be formulated without sacrificing bioequivalence. In particular, it was not apparent whether suitable binder/buffer systems could be identified which, in the absence of granulation, would serve to control nicotine release and absorption to approximately the same rate and extent as the mini-troche/lozenge of the Nicorette® NDA (as hereinafter defined). Furthermore, it was not apparent prior to the present invention whether such a tablet could be manufactured more economically by direct compression.

After repeated experimentation with different formulation approaches, the inventors have found it possible, using certain binder/buffering agent systems, to achieve direct compression tablets of the invention having the sensory benefits described above, which are not only more economical to produce, but are also demonstrated to be bioequivalent to the NICORETTE® 2 and 4 mg mini-troche/lozenges; as well as being comparable sized.

The present invention further provides a method for reducing withdrawal symptoms associated with smoking, and/or for obtaining a reduction of craving to smoke or use tobacco containing material and/or for providing a sense of smoking satisfaction without smoking, comprising the steps of replacing at least partly the tobacco containing material with a tablet of the invention, administering the tablet into the oral cavity of the subject and allowing the nicotine of the tablet to be released in the saliva in the oral cavity and absorbed by the subject into the systemic circulation of the subject.

Other features and advantages of the present invention will be apparent from the detailed description and the claims.

### BRIEF DESCRIPTION OF FIGURES

FIG. 1 shows the chemical name, structural formula, molecular formula, and molecular weight of Nicotine Polacrilex.

FIG. 2 is a Mean Linear Scale following 2 mg or 4 mg test tablets of the invention (referred to as the “Prototype Mini Lozenge” and the 2 mg or 4 mg Reference Products (referred to as the “Commercially Available Mini Lozenge” for PKAS1 (Baseline-Adjusted Concentrations) for the A) 2-mg Study and B) 4-mg Study”.

FIGS. 3-6 contain Tables 1-4, referred to in Example 4, as follows:
Table 1. “Summary of Plasma Pharmacokinetic Parameters of the Primary Endpoints for PKAS1 in the 2-mg and 4-mg Studies (Baseline-Adjusted PK Parameters)”;
Table 2. “Statistical Assessment of Bioequivalence for Test Tablet and Reference Product for hPKAS1 (Baseline-Adjusted PK Parameters)”;
Table 3. “Summary of Plasma Pharmacokinetic Parameters of the Secondary Endpoints for PKAS1 in the 2-mg and 4-mg Studies (Baseline-Adjusted PK Parameters)”;
Table 4. “Nonparametric Statistical Analysis of Tₘₐₓ for Nicotine for PKAS1 (Baseline-Adjusted PK Parameters)”.

### DETAILED DESCRIPTION

All publications including, but not limited to, patents and patent applications cited in this specification are incorporated herein by reference as though fully set forth.

The present invention may comprise, consist of, or consist essentially of the components set forth below, unless otherwise stated.

### Definitions

Unless otherwise stated, as used herein the term “a” or “an” includes one or more of the components modified.

“Bioequivalence” shall be understood to mean, in general, the absence of a significant difference in the rate and extent to which the active ingredient or active moiety in pharmaceutical equivalents or pharmaceutical alternatives becomes available at the site of drug action when administered at the same molar dose under similar conditions in an appropriately designed study (see 37 CFR § 320.10).

In one embodiment, the 2 mg and 4 mg nicotine intra-oral tablets of the invention are bioequivalent to the corresponding 2 mg or 4 mg Reference Product (as defined herein), wherein said bioequivalence is established by at least one pharmacokinetic parameter that is selected from (i) a confidence interval for mean AUC(₀₋ₜ) between about 80% and about 125%; (ii) a confidence interval for mean AUC (_{0-infin}) between about 80% and about 125%; (iii) a confidence interval for mean Cₘₐₓ between about 80% and about 125%; (iv) a confidence interval for mean Tₘₐₓ between about 80% and about 125%; and (v) combinations thereof;

In another embodiment, the 2 mg and 4 mg nicotine intra-oral tablets of the invention are bioequivalent to the corresponding 2 mg or 4 mg Reference Product, wherein said bioequivalence is established by at least one pharmacokinetic parameter that is selected from (i) a confidence interval for mean AUC(₀₋ₜ) between about 70% and about 143%; (ii) a confidence interval for mean AUC (_{0-infin}), between about 70% and about 143%; (iii) a confidence interval for mean Cₘₐₓ between about 70% and about 143%; (iv) a confidence interval for mean Tₘₐₓ between about 70% and about 143%; and (v) combinations thereof;
wherein, in the above embodiments, AUC₀₋ₜ is a measure of the total exposure of nicotine to the body up to the last sampling time; AUC_{0-infin} is a theoretical measure of the total exposure of nicotine to the body from administration until all the nicotine is eliminated; Cₘₐₓ is maximal plasma nicotine level; and Tₘₐₓ is time to reach maximal plasma nicotine concentration.

In the above embodiments, suitable combinations of pharmakinetic parameters may be selected from, e.g.: (i) and (ii); (i) and (iii); (ii) and (iii); and (i), (ii) and (iii); and each of the preceding combinations further combined with (iv).

Preferably, there is no substantial difference in pharmacokinetic parameters regardless of whether the nicotine tablet of the invention is administered to a subject in fed or fasted state.

“Intra-oral” with respect to the tablet of the invention is intended to refer to administration of nicotine into the systemic blood circulation by means of absorption of the nicotine by a tissue of the oral cavity.

“Organoleptic” is herein intended to refer to a feature that is discernable to the taste, mouth feel, smell, hearing and/or vision of the subject such as, but not limited to, flavor, cooling, burning, warming, tingling, bubbling, foaming, effervescing, heating, mouth watering, crunchiness, stickiness, physical form, texture, e. g., hardness, softness, roughness, and engravings.

“Orally disintegrating” refers to disintegration in presence of the small an amount of saliva normally resident in the mouth, in the absence of chewing or added liquids.

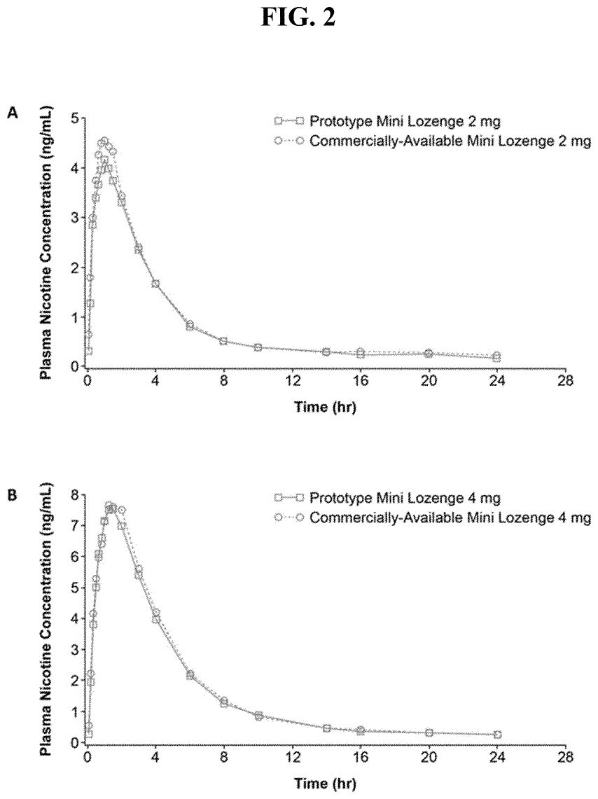

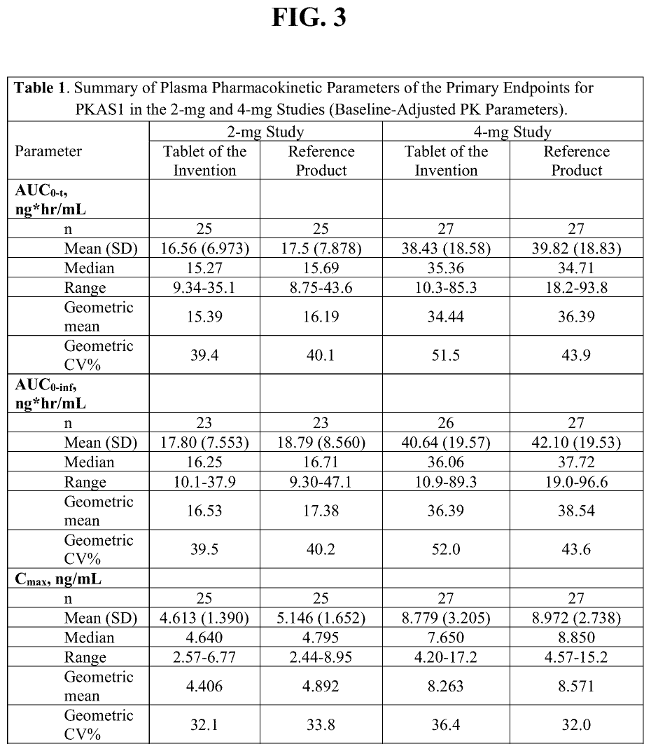

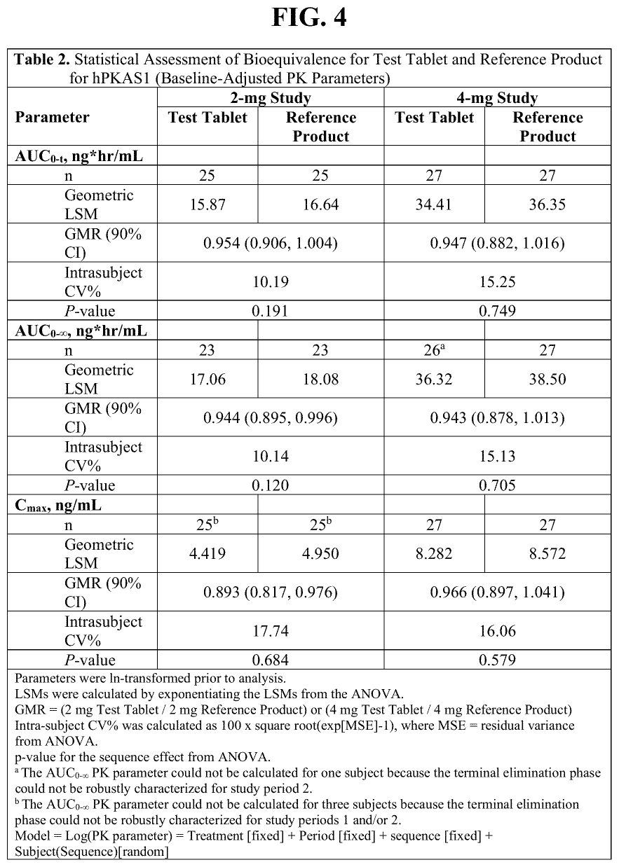

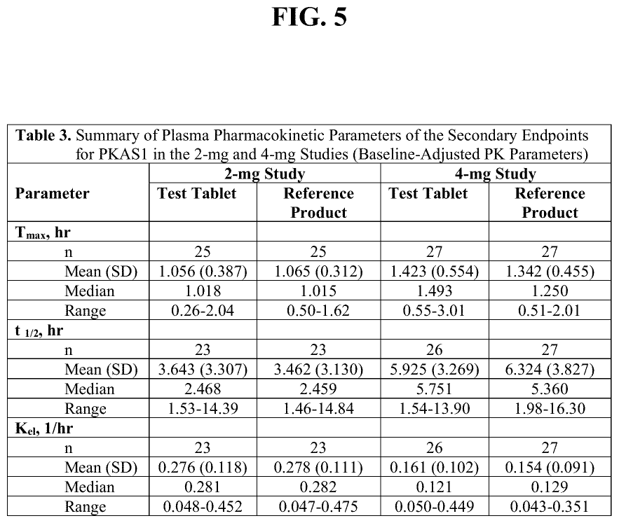

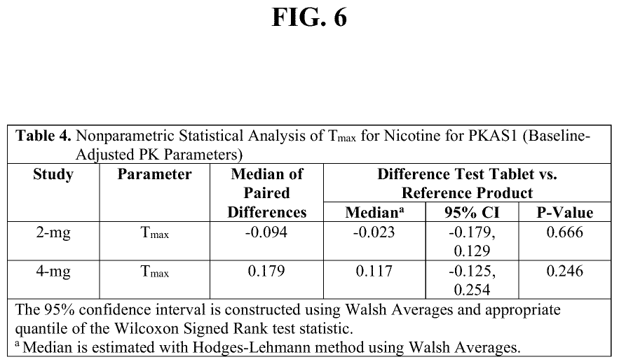

“Reference Product” as employed herein refers to the commercially available Nicorette® Nicotine Polacrilex (equivalent of 2 mg or 4 mg nicotine) mini-troche/lozenges which are the subject of New Drug Application (NDA) 22-360 (the redacted version of which became publicly available on or about May 18, 2009, said publicly available version, including all Supplements and Amendments thereon, being referred to herein collectively as the “Nicorette® NDA”, which is hereby incorporated by reference). Pharmacokinetic parameters, e.g., AUC₀₋ₜ, AUC_{0-infin}, Cₘₐₓ, and Tₘₐₓ, for the Reference Products are disclosed in the Nicorette® NDA as well as displayed on FIG. 2 and Tables 1-4 of Example 4 hereof. The Reference Products are described in Example 1 of U.S. Pat. Nos. 8,501,164 and 8,940,772, incorporated by reference.

“Tablet” as used herein shall refer to a compressed solid dosage form.

The present invention concerns a pharmaceutically acceptable oral tablet comprising an orally disintegrating, fast-releasing portion comprising a sensory marker/signal, and a more slowly disintegrating/eroding troche/lozenge portion from which nicotine is gradually released over time.

By “portion” is meant a part of the tablet that has been subjected to compression in the formation of the tablet, and thus would normally exclude film or other coatings.

In one embodiment, the tablet comprises one orally disintegrating portion and one troche/lozenge portion; and preferably each portion comprises a separate layer of the tablet. More preferably, the two portions comprise adjacent and preferably fully contiguous layers of the tablet, i.e. in the absence of a separating layer or film. Most preferably, the two portions comprise the two layers of a bilayer tablet. (It shall be understood, however, that a tablet of the invention may comprise more than one, e.g., 2 or more, of each of the orally disintegrating portion and the troche/lozenge portion.)

In a further embodiment, each portion comprises a layer of the tablet; the tablet is a bilayer tablet; and the top layer comprises the orally disintegrating portion, and the bottom layer comprises the troche/lozenge portion.

The tablet may be free of any external film coating or compression coating.

### Orally Disintegrating Portion

The orally disintegrating portion comprises a pharmaceutically acceptable formulation that rapidly disintegrates in the oral cavity. This portion is preferably free of nicotine active agent.

In one embodiment the orally disintegrating portion has a disintegration time in the oral cavity of about 3 minutes or less, and more preferably about 2 minutes or less. In particular embodiments, the disintegration time is no more than about 60, 55, 50, 45, 40, 35 or 30 seconds. The rapid disintegration in the mouth makes it possible for the sensory element/signal to be released without any accompanying liquid.

Disintegration time may be measured in accordance with USP monograph 701. FDA Guidelines indicate that the term “orally disintegrating tablet” be used for solid oral preparations having an in vitro disintegration time of approximately 30 seconds or less when tested according to this method. Formulations which disintegrate in under 3 minutes when tested under this method may be considered to be “orodispersible” in accordance with the European Pharmacopoeia (8^{th} ed.). The orally disintegrating portion of the tablets of the invention may be considered to be orally disintegrating or orodispersible, consistent with FDA usage or the European Pharmacopeia.

### Sensory Marker/Signal

The sensory marker/signal may be any pharmaceutically acceptable, preferably therapeutically inactive, ingredient that exerts an organoleptic effect.

The organoleptic effect is preferably an effect on taste (and optionally also having an effect on, e.g., smell). Suitable flavorants exerting, in appropriate amounts, an effect on taste include, but are not limited to, mint, peppermint, wintergreen, spearmint, menthol, vanillin, chocolate, coffee, cinnamon, clove, tobacco, citrus and fruit flavors, and combinations thereof.

Most preferably the flavorant is a natural or synthetic flavor which, alone or combined with sweetening agents, exerts a breath-freshening effect, and optionally also a taste-masking effect on nicotine. It has been determined through consumer testing that flavorants having the desired breath-freshening and optional taste-masking effect are mint and/or fruit flavors, such as are available from Firmenich.

Suitable sweeteners which may be used to augment the sensory marker/signal include natural and synthetic sweeteners, i.e. any form of carbohydrates suitable for use as sweetener, as well as so-called artificial sweeteners such as saccharin, sodium saccharin, aspartame, acesulfame, potassium acesulfame, thaumatin, glycyrrhizin, sucralose, dihydrochalcone, alitame, miraculin, monellin, stevside and neotame, and combinations thereof.

Preferred sweeteners comprise synthetic sweeteners, such as sucralose, acesulfame potassium, and combinations thereof. The combination of equal amounts by weight of sucralose and acesulfame potassium is particularly preferred.

The amount of sensory marker/signal in the orally disintegrating portion may be, e.g., about 0.5 to about 10 wt. %, preferably from about 3 to about 7 wt. %, based on that portion.

Total sweetener may be provided in a total amount of about 0.5 to about 10 wt. %, e.g., about 1 to about 5 wt. %, based on the orally disintegrating portion.

A preferred combination of flavor and sweetener comprises: mint or fruit flavor, with sweetener comprising sucralose and acesulfame potassium.

### Excipients for Orally Disintegrating Portion

The orally disintegrating portion may comprise at least about 50 wt. %, and preferably at least 70 wt. %, e.g., up to about 90 wt. %, of compressible excipient(s), based on the weight of the orally disintegrating portion.

Preferably, at least one compressible excipient is a “disintegration promoting excipient” and at least one compressible excipient serves as a bulking agent/filler.

Disintegration promoting excipients are compressible excipients which enable the manufacture of compressed tablets which have an oral disintegrating time of less than about 3 minutes. The disintegration promoting excipient may be a single substance (referred to herein as a “disintegrant”) or a mixture of disintegrants.

Suitable disintegrants include, but are not limited to, “super-disintegrants” such as crospovidone, croscarmellose sodium, sodium starch glycolate, and mixtures thereof. Superdisintegrants act by absorbing saliva, leading to mechanical swelling and rapid tablet disintegration. Other disintegrants include low-substituted hydroxypropyl cellulose (L-HPC), pregelatinized starch, microcrystalline cellulose, and mixtures thereof.

Alternatively, the disintegration promoting excipient may comprise a mixture of disintegrants and other substances such as one or more bulking agents/fillers and binders (said mixture being collectively referred to as a “disintegration promoting excipient pre-mix”) wherein said mixture functions as a disintegration promoting excipient.

Suitable bulking agents/fillers for use in a disintegration promoting excipient pre-mix include carbohydrate or non-carbohydrate fillers. A carbohydrate is preferred, although non-carbohydrate bulking agents/fillers may be present in the alternative or in admixture with a carbohydrate. The term “carbohydrate” shall be understood to includes sugars (e.g. monosaccharides, disaccharides and oligosaccharides, such as maltose and dextrin) as well as derivatives thereof, especially polyhydric alcohol derivatives such as mannitol, xylitol and sorbitol, and combinations thereof.

Mannitol is preferred for its sweetness and pleasant mouthfeel. Examples of non-carbohydrate bulking agents/fillers include (anhydrous) dibasic calcium phosphate, tribasic calcium phosphate, and magnesium aluminometasilicate, and combinations thereof.

An example of a commercially available disintegration promoting excipient pre-mix is F-melt® (Fuji Chemicals), which is available as F-melt® Type C and F-melt® Type M.

F-melt® Type C comprises a mixture of D-mannitol (about 62 to about 68 wt. %, e.g., about 65-66%), xylitol (about 4 to about 6 wt. %, e.g., about 5%), dibasic calcium phosphate anhydrous (DCPA) (e.g., about 3 to 5 wt. %, e.g., about 4 wt. %), crospovidone (e.g., about 7 to about 9 wt. %, e.g., about 8 wt. %) and microcrystalline cellulose (e.g., about 16 to about 20 wt. %, e.g., about 17 wt. %) (Fuji Chemicals Certificate of Analysis, Jul. 7, 2015) (all percentages being based on the disintegration promoting excipient pre-mix composition).

F-melt® Type M comprises mannitol, xylitol, magnesium aluminometasilicate (Neusilin®) crospovidone and microcrystalline cellulose in spherical particles having high flowability.

Other disintegration promoting excipient pre-mixes include Ludiflash® (BASF Fine Chemicals), comprising mannitol (90%), crospovidone (Kollidon® CL-SF) (5%) and polyvinyl acetate (Kollicoat® SR 30D) (5%); Galen1Q®, comprising a disaccharide alcohol in a 3:1 ratio of 6-O-oD-glucopyranosyl-D-sorbitol and 1-O-a-D-glucopyranosyl-D-mannitol dihydrate; Prosolv® ODT G2 (JRS Pharma), comprising microcrystalline cellulose, colloidal silicon dioxide, mannitol, fructose and crospovidone; Pharmaburst®, comprising mannitol, sorbitol, crospovidone, croscarmellose sodium and colloidal silicon dioxide; and Smart-Ex® (Shin-Etsu) comprising mannitol, L-HPC and polyvinyl alcohol (see, e.g., U.S. Pat. No. 10,729,655 at col. 4,1. 16 et seq.).

The above disintegration promoting excipient pre-mixes are generally co-processed by spray-drying to form particles in which the individual components of the pre-mix are rendered mechanically inseparable.

The tablets of the invention contain a sufficient amount of at least one disintegration promoting excipient or excipient premix in order for the immediately disintegrating portion of the tablet to disintegrate within a sufficiently short time as previously described, preferably 60 seconds or less, following administration. However, as will be apparent to the skilled worker, the amount of the at least one disintegrating excipient or excipient premix must not be so high as to cause sticking or picking of the formulation on tablet punches.

F-melt® Type C, in particular, in an amount of about 70 to about 80 wt. %, based on the orally disintegrating portion, was found to support manufacturability with good flow and an acceptable disintegration time. Advantageously, it was found that disintegration time of less than 60 seconds could be achieved by using this premix without added disintegrants or super-disintegrants.

Bulking agents/fillers. The rapidly disintegrating portion includes from about 5% to about 50% by weight, preferably about 5 to about 25% by weight, of one or more bulking agents/fillers, apart from any bulking agent/filler contained in a disintegration promoting excipient pre-mix (all percentages being based on the orally disintegrating portion).

Suitable bulking agents/fillers are as previously described in connection with the disintegration promoting excipient pre-mix. A preferred bulking agent/filler for use in the immediately disintegrating portion apart from any disintegration promoting excipient pre-mix, comprises mannitol.

Combinations of F-melt® Type C, in an amount of about 70-about 80 wt. %, and mannitol, in an amount of about 10 to about 20 wt. % (based on the orally disintegrating portion), have been found particularly suitable in providing rapid disintegration combined with good manufacturability.

Lubricant. The fast disintegrating portion of the tablet of the invention may comprise one or more lubricants. Suitable lubricants include, for example, magnesium stearate, stearic acid, palmitic acid, calcium stearate, carnauba wax, hydrogenated vegetable oils, mineral oil, polyethylene glycols and sodium stearyl fumarate. In one embodiment, the lubricant is magnesium stearate.

Magnesium stearate in an amount of from about 0.5 to about 2.5 wt. % (e.g., about 1 wt. %) based on the orally disintegrating portion, was found to provide optimal flowability and disintegration time.

### Additional Ingredients

Additional conventional ingredients may include known preservatives, anti-oxidants, coloring agents and stabilizers. In a preferred embodiment, the orally disintegrating portion of the tablet of the invention is free of preservatives and/or stabilizers and/or anti-oxidants.

The at least one orally disintegrating portion is preferably free of alkaline buffering agent, as well as being preferably free of nicotine.

The orally disintegrating portion of the tablet most preferably comprises, or consists essentially of, the ingredients listed for this portion in Example 1. “By consisting essentially of” is meant that any additional ingredients will have an insubstantial effect on the disintegration rate of the orally disintegrating portion. The proportions for any of the ingredients indicated in Example 1 may be altered in line with the proportions described elsewhere herein.

### Nicotine-Containing Troche/Lozenge Portion

### Nicotine Active Agent

Nicotine is a natural alkaloid obtained from the dried leaves and stems of the Nicotiana tabacum and Nicotiana rustica. It is a hygroscopic, oily, colorless or pale yellow liquid, which is miscible with water in its neutral amine form between 60° C. and 210° C.

Nicotine, 3-(1-methyl-2-pyrrolidinyl) pyridine, molecular formula C₁₀H₁₄N₂, is a bicyclic compound containing a chirogenic center at the 2′-position of the pyrrolidine moiety, and existing as two enantiomers: R-(+)-nicotine and S-(−)-nicotine. The (S)-form, the most active enantiomer, is reported to be the only naturally occurring form. The molecular weight is 162.26 g/mol.

The term “nicotine active agent” as used herein is intended to include nicotine, (S)-3-(1-methyl-2-pyrrolidinyl)-pyridine, in its base form, including synthetic nicotine as well as nicotine extracts from tobacco plants or parts thereof, such as the genus Nicotiana, alone or in combination; and pharmaceutically acceptable salts thereof.

Examples of possible acids useful for nicotine salt formation include (in ratio of acid to nicotine) include:Formic 2:1, Acetic 3:1, Propionic 3:1, Butyric 3:1, 2-Methylbutyric 3:1, 3-Methylbutyric 3:1, Valeric 3:1, Lauric 3:1, Palmitic 3:1, Tartaric 2:1, Citric 2:1, Malic 2:1, Oxalic 2:1, Benzoic 1:1, Gentisic 1:1, Gallic 1:1, Phenylacetic 3:1, Salicylic 1:1, Phthalic 1:1, Picric 2:1, Sulfosalicylic 1:1, Tannic 1:5, Pectic 1:3, Alginic 1:2, Hydrochloric 2:1, Chloroplatinic 1:1, Silicotungstic 1:1, Pyruvic 2:1, Glutamic 1:1, and Aspartic 1:1.

Pharmaceutically acceptable salts of nicotine include, e.g., the monotartrate, hydrogen tartrate (also called bitartrate or bitartrate dihydrate), citrate, malate, and/or hydrochloride.

The term, “nicotine active agent” shall also be understood to also include nicotine derivatives, nicotine isomers and prodrugs of nicotine.

The term, “nicotine derivative” shall be further understood to include nicotine cation exchangers, nicotine inclusion complexes and nicotine in any non-covalent binding interaction, nicotine bound to zeolites, and nicotine bound to cellulose or starch micro spheres, as are well-known in the art.

Alternatively, the “nicotine active agent” may be selected from nicotine metabolites such as nicotine N′-oxide, nornicotine, (S)-nicotine-N-.beta.-glucuronide and mixtures, isomers, salts and complexes thereof.

In the compositions of the invention, the nicotine active agent is nicotine in a form that serves to retard release of nicotine into the oral cavity. A preferred controlled release form of nicotine is nicotine bound to a cation exchange resin. Suitable cation exchangers are well-known, including, for example, divinylbenzene-methacrylic acid polymer resins such as Amberlite IRC 50, Amberlite IRP64 and Amberlite IRP64M (Rohm & Haas); as well as Doshion P551 (Doshion Poly Science), and Purolite C115HMR (Purolite); styrene-divinylbenzene resins such as Amberlite IR118, Amberlite IRP 69, Amberlite IRP 69M, and Amberlite IR 120 (Rohm & Haas); Dowex 50W (Dow Chemical); and Duolite C-25 (Chemical Process Co.); and phenolic polymer resins such as BIO-REX 40 (BIO-RAD Lab) and Duolite C-3 and C-10 (Chemical Process Co.).

Most preferably, the nicotine active agent comprises Nicotine Polacrilex (NPA), also known as Nicotine Resinate, which is a weak carboxylic cation-exchange resin prepared from a polymethacrylic acid resin, such as e.g., the aforementioned Doshion P551, Amberlite IR64, or Purolite C115HMR.

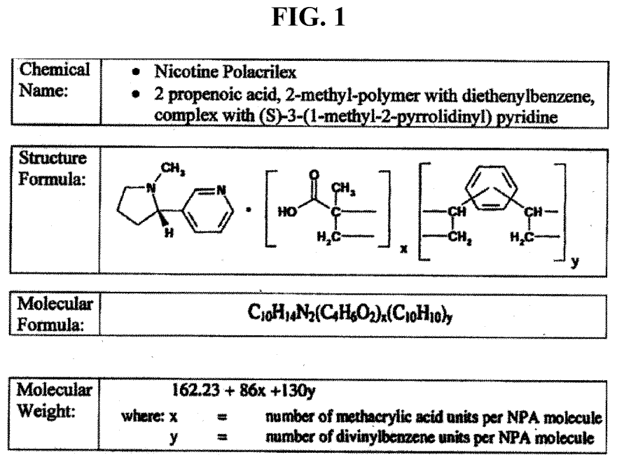

NPA has the chemical name, structural formula, molecular formula, and molecular weight as shown on FIG. 1.

Nicotine forms an ionic complex with polacrilex which is stable and water insoluble. Once administered, the release of nicotine from the polymer resin complex occurs through an ionic exchange process with counter ions that also become available through dissolution in the oral cavity. This results in the release of free nicotine from the water insoluble resins.

Nicotine Polacrilex (NPA) USP/EP is commercially available from various manu-facturers as 20% (200 mg/g) nicotine powder in water. The nicotine powder is over 99% pure, and is preferably 99.99% pure.

### Amount and Distribution of the Nicotine in the Troche/Lozenge Portion

The troche/lozenge portion according to the invention is formulated to provide the subject with a therapeutically effective amount of nicotine. The therapeutic effect may be to provide a sense of smoking satisfaction without smoking. Another therapeutic effect of the administered nicotine in any form may be a reduction of the craving to smoke or use tobacco. The therapeutic effect may also be a combination of reduction of said craving and providing a sense of smoking satisfaction without smoking. The amount of the nicotine should be sufficient to provide such an effect in a subject. This amount may, of course, vary from person to person.

The tablets of the invention comprise nicotine active agent in any amount. A unit dose tablet will comprise sufficient nicotine active agent to provide nicotine in a therapeutically effective amount, which effective amount may be in the range of, for example, from 0.05 mg to 12 mg. In various embodiments, a unit dose amount may be 0.05, 0.5, 1, 2, 3, 4, 5, 6, 7, 8, 9, 10, 11 or 12 mg, preferably in the range of 0.1-6 mg, more preferably in the rage of 1-6 mg, and most preferably in the range of 2-5 mg, e.g., 2 mg or 4 mg, all such amounts being based on nicotine. The nicotine active agent will be mixed with the inactive ingredients of this portion.

### Buffering Agents

The nicotine active agent-containing troche/lozenge portion of the tablet will also generally comprise one or more alkaline buffering agent/s to facilitate nicotine administration.

Absorption of nicotine from the oral cavity to the systemic circulation is dependent on the pH of the saliva (normally, 6.4 to 7.0) and the pKa of nicotine, which is about 7.8. The amount and type of buffering agent will affect the pH of the saliva and hence the release of nicotine base, which is the form predominantly absorbed. The buffering is intended to achieve a transient buffering during disintegration or dissolution of the troche/lozenge portion of the tablet, after which the pH gradually returns to its normal value.

Suitable alkaline buffering agents include but are not limited to alkali metal carbonates and bicarbonates such as sodium carbonate (especially, anhydrous sodium carbonate), sodium bicarbonate (i.e. sodium hydrogen carbonate), potassium carbonate and potassium bicarbonate and the like; alkali earth metal carbonates and bicarbonates such as magnesium carbonate, magnesium bicarbonate, calcium carbonate, calcium bicarbonate and the like; as well as potassium phosphate, dibasic sodium phosphate, sodium borate, sodium aluminate, magnesium oxide, magnesium hydroxide, and magnesium silicates, and combinations of the foregoing.

Preferably, the type and amount of buffering agent(s) will be selected to achieve a salivary pH in the range of about 7.1 to about 8.3, more preferably in the range of about 7.5 to about 8.2, even more preferably about 7.5 to about 7.8.

A preferred buffering agent comprises the combination of sodium carbonate (esp. anhydrous sodium carbonate) and sodium bicarbonate.

Most preferably, the anhydrous sodium carbonate and the sodium bicarbonate are present in a weight percent relationship of about 10:1 to about 7:1, preferably about 8:1; and the total amount of buffer is in a range of about 2 wt. % to about 12 wt. %, especially about 4 wt. % to about 10 wt. %, based on the weight of the troche/lozenge portion of the tablet.

Binders

Binders in the nicotine-containing troche/lozenge portion are selected to control disintegration, dissolution or erosion of the nicotine-containing troche/lozenge portion in the buccal cavity. Preferred binders are binders which alone or in combination form an at least partially hydrophilic matrix.

Exemplary rate-control binders are selected from, but are not limited to, cellulose and its derivatives including ethyl cellulose, hydroxypropyl methyl cellulose (Hypromellose), hydroxypropyl cellulose, methylcellulose, hydroxyethyl cellulose, and carboxymethyl cellulose, and combinations thereof; gelatin, liquid glucose; starch and its derivatives (e.g. corn starch); sugars, polyvinyl pyrrolidone, agar, acacia, alginic acid or a salt thereof such as sodium alginate and potassium alginate, carbomer, carrageenan, chitosan, tragacanth, xanthan gum, guar gum, inulin, pectin, polycarbophil or salt thereof such as calcium polycarbophil, polyethylene glycol, polyethylene oxide, polyvinyl alcohol, and pullulan; and combinations of the foregoing.

A preferred hydrophilic matrix-forming binder is Hypromellose, especially having viscosity of about 80-120 mPa·s (2% in water at 20 C). Suitable grades include HPMC K100LV Premium CR (Dow) and Metolose 90SH-100SR (Shin-Etsu).

The total amount of binder, may for example, be in the range of about 1 to about 20 wt. %, e.g., about 2 to about 15 wt. %, based on the weight of the troche/lozenge portion.

The troche/lozenge portion of the tablets of the invention comprises the nicotine active agent in an at last partially hydrophilic matrix that is designed to hydrate and dissolve slowly in the buccal cavity over a period of about 10 to about 15 minutes. When the nicotine active agent is NPA, absorption of moisture by the hydrophilic matrix triggers the dissociation of nicotine monocation from the insoluble polacrilex resin. Although NPA complex is non-reactive in its bound form, nicotine active is easily released in aqueous media under optimal pH conditions and the active retains all of its pharmacological activities. Thus in the presence of buffer, nicotine monocation converts to nicotine base, which is readily absorbed by the oral mucosa (see further, Chen, L. and Kamath, S., “CH. 16. ASAP Application: Nicotine Lozenges,” in Accelerated Predictive Stability, Fundamentals and Pharmaceutical Industry Practices, Elsevior Inc., 2018.)

In the presence of suitable amounts and combinations of binder and buffer, the dissolution rate of the troche/lozenge portion and absorption of nicotine is such that, in one embodiment, the nicotine will reach similar plasma levels about 30 minutes after administration as would be attained by the treated subject when smoking a cigarette.

It has been a particular object of this invention to identify suitable binder/buffering agent systems that work in concert to control nicotine release while elevating pH for a sufficient time so that the rate and extent of nicotine absorption by the blood from a tablet of the invention is equivalent to that of the Reference Products of the Nicorette® NDA, despite the structural differences between the tablet of the invention and the Reference Products.

To that end, the inventors have discovered that the use of a binder system comprising the combination of Hypromellose and xanthan gum in relative amounts by weight of about 0.75:1 to about 3:1, especially about 1.1 to about 1.5:1, preferably in combination with a buffer system comprising sodium carbonate anhydrous, and sodium bicarbonate in relative amounts of about 10:1 to about 7:1, especially about 8 to 1, by weight, in the troche/lozenge portion, advantageously facilitates release and absorption of nicotine at a rate and an extent which is therapeutically effective, and which furthermore, is Bioequivalent to that of the Reference Products.

### Other Ingredients of the Troche/Lozenge Portion

Bulking agent/Filler

Suitable bulking agents/fillers for use in the troche-lozenge portion are as previously described in connection with the orally disintegrating portion.

In particular, mannitol (especially, Pearlitol® 200 SD (Roquette) is suitably used as a bulking agent/filler. Mannitol or other bulking agent/fillers may be present in a total amount of from about 60 to about 90 wt. %, and preferably from about 70 to about 80 wt. %, based on the nicotine active agent-containing troche/lozenge portion.

The nicotine active agent-containing troche/lozenge portion is preferably free of glucosides of mannitol, such as isomalt.

Additionally, flavors and/or sweeteners may be included in the nicotine active agent-containing portion. The same or different flavors may be used for the orally disintegrating and troche/lozenge portions.

Flavors may be present in an amount of from about 0.5 to about 10 wt. %, e.g., from about 3 to about 7 wt. %, based on the troche/lozenge portion.

Total sweetener may be provided in a total amount of about 0.5 to about 10 wt. %, e.g., about 1 to about 5 wt. %.

A preferred combination of breath-freshening/taste-masking flavor and sweetener for use in the troche/lozenge comprises: mint or fruit flavor (Firmenich) (e.g., about 3 to about 7 wt. %); sucralose (e.g., about 0.5 to about 2.5 wt. %) and acesulfame potassium (e.g., about 0.5 to about 2.5 wt. %) (all weight percentages being based on the weight of the nicotine-containing troche/lozenge portion).

The use of a flavorant, such as mint, in both the orally disintegrating portion as well as the slower-releasing troche/lozenge portion, provides the sensory burst of intense flavor previously described followed by a prolonged period of flavor as the product dissolves.

Additional ingredients may include lubricants, as previously described in connection with the orally disintegrating portion, as well as preservatives, anti-oxidants, coloring agents, and stabilizers. In a preferred embodiment, the nicotine-active agent containing troche/lozenge portion of the tablet is free of preservatives, stabilizers or anti-oxidants.

It has been found that a lubricant such as magnesium stearate, in an amount of at least about 2 wt. % but generally no more than 3 wt. %, based on the troche/lozenge portion, is particularly effective in controlling striations and material sticking during compression.

The composition is found to have sufficient flowability without requirement for silicon dioxide.

Accordingly, embodiments of compositions of the invention comprise the following ingredients in the respective orally disintegrating and troche/lozenge portions, in the indicated amounts:

Orally disintegrating portion:¹

| Ingredient | Amount |
|---|---|
| sensory marker/signal | from about 0.5 to about 10 wt. %, preferably from about 3 to about 7 |
| | wt. % |
| optional sweetener | from about 0.5 to about 10 wt. %, e.g., from about 1 to about 5 wt. % |
| compressible excipient | at least about 50 wt. %, e.g., from about 50 to about 90 wt. %, e.g., from |
| | about 70 to about 80 wt. %² |
| bulking agent/filler | from about 5 wt. % to about 50 wt. % by weight, preferably from about |
| | 5 wt. % to about 25 wt. % |

¹all weight percentages based on weight of the orally disintegrating portion
²e.g., F-melt ® Type C, in an amount of from about 70 to about 80 wt. %, and mannitol, in an amount of from about 10 wt. % to about 20 wt. %

Troche/Lozenge portion³:

| Ingredient | Amount |
|---|---|
| Nicotine active agent | sufficient to provide from 0.05 mg to 12 mg nicotine per unit dose (i.e. |
| | one tablet) |
| Buffer system | total from about 2 wt. % to about 12 wt. %, e.g., from about 4 wt. % to |
| | about 10 wt. %⁴ |
| Binder | From about 1 to about 20 wt. %, e.g., about 2 to about 15 wt. %⁵ |
| Bulking agent/fdler | from about 60 to about 90 wt. %, preferably from about 70 to about 80 |
| | wt. %, |

³all weight percentages based on weight of the troche/lozenge portion
⁴e.g., comprising sodium carbonate anhydrous and sodium bicarbonate in relative amounts by weight of about 10:1 to about 7:1)
⁵e.g., comprising hypromellose and xanthan gum in relative amounts by weight of about 0.75:1 to about 3:1, especially about 1.1 to about 1.5:1

### Method of Manufacture

Advantageously, the tablets of the invention are found to be suited to be prepared by a process of direct compression, i.e. in the absence of wet or dry granulation, from intermediate compositions in the form of powders essentially free of granules.

The intermediate compositions comprising the respective orally disintegrating portion and nicotine-containing troche/lozenge portion, are each prepared by blending the ingredients of each portion, with optional sieving as described in Example 1.

In an embodiment of the invention wherein the tablet is a bilayer tablet, the intermediate compositions making up each of the layers are provided to respective sides of a tablet press (the disintegrative portion preferably comprising the upper layer, and the nicotine-containing portion preferably comprising the lower layer) for an optional pre-compression step, which is then followed by combining of the layers and compression of the layers to form the tablet of the invention.

Target pre-compression for the respective layers is generally set for about 1-1.5 kN. Preferred compression roll force is no more than 20 kN. Tablet hardness (SCU) is preferably in the range of about 5.0 to about 11.0, more preferably from about 5.0 to about 9.0. Friability of the resulting tablets does not exceed 1.0%.

Mini-lozenge format

The tablet of the invention preferably has overall dimensions and/or weight consistent with the NICORETTE® mini-troche/lozenge format.

Accordingly, in one embodiment, the tablet of the invention has a total weight of from about 200 to about 300 mg, more preferably from about 250 to about 300 mg, even more preferably from about 255 to about 285 mg. Preferably, for a bilayer tablet according to the invention, the orally disintegrating layer has a weight of from about 60-85 mg; and the nicotine active agent-containing troche/lozenge layer has a weight of about 180 to about 225 mg.

A tablet of the invention which is Bioequivalent to the Reference Product comprises an oval tablet having a length of from about 0.4 to about 0.5, e.g., about 0.44; a breadth of from about 0.2 to about 0.25, e.g., about 0.23; and a maximum thickness of about 0.18 to about 0.22, e.g., about 0.19 to about 0.21, e.g., 0.20 (in inches). Similarly, the Reference Product is an oval tablet having a length of about 0.4 inch; a breadth of about 0.21 inch; and a maximum thickness of about 0.21 inch.

### Methods of the Invention

The invention also provides methods employing the tablets of the invention for nicotine replacement therapy.

In particular, the invention comprises a method for reducing withdrawal symptoms associated with smoking or use of a tobacco containing material; and/or for obtaining a reduction of the urge or craving to smoke or use tobacco containing material; and/or for providing a sense of smoking satisfaction without smoking, in a mammalian (i.e. human) subject in need thereof, comprising the steps of replacing at least partly the tobacco containing material with a tablet of the invention, administering the tablet into the oral cavity of the subject and allowing the nicotine of the tablet to be released in the saliva in the oral cavity and absorbed by the subject into the systemic circulation of the subject.

Suitable directions for self-administration by a subject are as follows:

If the subject smokes his/her first cigarette within 30 minutes of waking, then the subject should use the 4 mg unit dose tablet; and if the subject smokes his/her first cigarette more than 30 minutes after waking, then the subject should use the 2 mg unit dose tablet; in either case according to the following 12 week schedule:

| NRT Therapy, Weeks | Dose Regimen |
|---|---|
| 1-6 | 1 tablet every 1-2 hours |
| 7-9 | 1 tablet every 2-4 hours |
| 10-12 | 1 tablet every 4-8 hours |

To improve chances of quitting smoking, the subject should use at least 9 tablets per day for the first 6 weeks. No more than 5 tablets should be used in 6 hours.

Dosing of the tablet of the invention is symptom dependent, with 20×4 mg tablets typically being the maximum daily dose administered to a subject.

The following examples are illustrative of the invention but are not intended to be limitative thereof.

### Example 1

A unit dose tablet having a rapidly disintegrating portion containing mint flavor (Firmenich) as the sensory marker/signal, and a troche/lozenge portion containing 2 mg nicotine, as nicotine polacrilex, was prepared from the following ingredients:

Nicotine 2 mg bilayer tablet
Orally disintegrating layer

| | | % w/w |
|---|---|---|
| Excipient | Function | in OD layer |
| F-Melt, Type C | Disintegration Promoting | 70-80 |
| | Excipient pre-mix | |
| Mannitol | Bulking agent/Filler | 10-20 |
| Mint Flavor | Sensory marker/signal | 3-7 |
| Sucralose | Sweetener | 0.5-2.5 |
| Acesulfame Potassium | Sweetener | 0.5-2.5 |
| Magnesium stearate | Lubricant | 0.5-2.5 |
| FD&C Blue #2 Indigo Carmine | Color | 0.5-2.5 |
| Aluminum Lake | | |

| | |
|---|---|
| Total | 100 |

Active layer comprising Nicotine Polacrilex*,
10.70 mg (equiv. 2 mg nicotine)

| | | % w/w in |
|---|---|---|
| Excipient | Function | active layer |
| Mannitol | Bulking agent/Filler | 70-80 |
| Mint Flavor | Flavor | 3-7 |
| Hypromellose | Binder | 2-8 |
| Sodium Carbonate Anhydrous | Buffer | 3-6 |
| Xanthan gum | Binder | 2-5 |
| Magnesium stearate | Lubricant | 2-3 |
| Sucralose Powder | Sweetener | 1-3 |
| Acesulfame Potassium | Sweetener | 1-3 |
| Sodium Bicarbonate Powder | Buffer | 0.25-1.75 |
| FD&C Blue Dye #2 (Indigo | Coloring agent | 0.5-2.5 |
| Carmine Al Lake) | | |

| | |
|---|---|
| Total | 100 |

*nominally 20% w/w nicotine

### Part (A) Orally Disintegrating Layer

### I. Screening

(a) Mint flavor (all flavors supplied by Furmanich), sucralose, mannitol (Pearlitol® SD200, Roquette) and acesulfame potassium were individually screened through ASTM #20 mesh screen (840 μm) or equivalent mesh.
(b) F-melt® Type C and FD&C Blue #2 were combined and screened through ASTM #40 mesh screen (420 μm) or equivalent mesh.
(c) Magnesium stearate was screened through ASTM #30 mesh screen (595 μm) or equivalent mesh.

### II. Blending

(a) Pre-lubrication. The material from I.(a) and I.(b) were transferred to a 5 cubic foot V-blender and blended at 25 rpm for 15 min.
(b) Lubrication. The material from I.(c) was transferred to the blender and mixed at 25 rpm for 4 min.
Part (B) Nicotine active agent-containing layer.

### I Screening.

(a) Mannitol was screened through ASTM #20 mesh (840 μm) screen or equivalent mesh; and divided into two equal parts, (i) and (ii).
(b) NPA was dispensed.
(c) Mint flavor, sucralose, acesulfame potassium, Hypromellose (Methocel®K100LV), xanthan gum, sodium carbonate anhydrous, and sodium hydrogen carbonate were individually screened through ASTM #20 mesh or equivalent mesh.
(d) Magnesium stearate was screened through ASTM #30 mesh (840 μm) screen or equivalent mesh.
(e) The materials from I.(a)(i), I(b), I.(c) and I.(a)(ii) were sequentially transferred to a 10 cubic foot V-blender and mixed at 19.1 rpm for 20 minutes.

### II. Lubrication.

(a) The material from I(d) was added to the blender and mixed for lubrication at 19.1 rpm for 5 minutes.
(b) The lubricated nicotine layer blend was transferred into double polythene lined drum(s) and labeled accordingly.

### Part (C) Compression

A Manesty 700 tablet press equipped with 0.2250×0.4350 inch oval shaped punches with “4” debossed on the upper face and a plain lower face, was used.

The bottom, nicotine layer blend obtained from step II(b) of Part (B) was transferred to side 1, and the top, disintegrative layer blend from Part (A), step II (b) was transferred to side 2, of the hopper of the tablet press.

Bilayer tablets were compressed at approximately 1KN pre-compression force and a main compression force of 7-16 KN which resulted in compact hardness in the range of 5-9 SCU. Tablet friability was less than 1%.

The product was a dual layer lozenge/troche of 270 mg with one side, white to off-white, and the other side, blue with debossed “4”.

### Example 2

A 4 mg nicotine as Nicotine Polacrilex tablet was prepared similarly as described in Example 1.

### Example 3

2 and 4 mg nicotine as Nicotine Polacrilex tablets were prepared similarly as described in Example 1, wherein in each case the orally disintegrating layer comprised Fruit flavor; and the nicotine-containing layer comprised Mint flavor.

### Example 4—Pharmacokinetic Characterization

Abstract. Two single-center, open-label, randomized, four-treatment, two-period, crossover studies were conducted to evaluate, respectively, 2 mg and 4 mg nicotine as Nicotine Polacrilex tablets prepared as described in Examples 1 and 2 (hereinafter, the “test tablets”). Methods: Two randomized, open-label, cross-over studies were conducted to evaluate either the 2- or 4-mg dose level. Heavy smokers in otherwise good health were randomly assigned to one of two treatment sequences: the test tablet followed by the corresponding Reference Product, or the converse. After a 5- to 7-day washout period, subjects crossed over to receive the other study treatment. Blood sampling occurred pre- and post-dose nicotine and was assessed using a validated solid-phase extraction with ultra-high-performance liquid chromatography and tandem mass spectrometry. The primary endpoint was bioequivalence as determined by maximal plasma nicotine concentration (Cₘₐₓ) and the extent of nicotine absorption (AUC₀₋ₜ and AUC_{0-∞}). The secondary endpoints included the time to Cₘₐₓ (Tₘₐₓ), half-life, the elimination constant (Kₑₗ), and safety.

### Study Subjects

All subjects were considered heavy smokers, defined as having smoked cigarettes for at least 12 months with the first cigarette of the day smoked within 30 minutes of waking. Subjects were in otherwise good health, with key inclusion criteria including age 19 to 55 and body mass index (BMI) of 19 to 28 kg/m². Subjects were excluded if they had an acute or chronic medical or psychiatric condition or laboratory abnormality that many increase their risk of taking the study treatments; current pregnancy or breastfeeding; history of regular alcohol use of more than 14 drinks per week; a positive illicit drug screen including tetrahydrocannabinol (THC); and, drinking more than 5 cups of coffee or tea per day. Subjects were not allowed to use NRT, tobacco products other than cigarettes, or electronic cigarettes within 21 days of the first study session. Other key exclusion criteria included use of prescription or experimental drugs within 2 weeks or 5 half-lives prior to the first dose of the study drug, except for contraception or hormone replacement therapy, and any known or suspected allergies/intolerances to the study treatment, or any medical condition that could alter the absorption, distribution, metabolism, or excretion of the nicotine. Sexually active subjects of childbearing potential were required to agree to appropriately using a highly effective method of contraception during the study period and for at least 5 days after the last treatment dose. Such contraception was defined as a method with a less than 1% failure rate per year when used correctly and consistently. In addition, subjects of childbearing potential underwent a serum pregnancy test during the screening visit and at check-in prior to the first and second treatment sessions.

### Treatment Periods

Subjects underwent a screening visit prior to the studies. For both studies, subjects were confined to the test facility for the duration of each treatment period for a total of 60 hours each. Each confinement period included a baseline check-in visit with randomization, pretreatment phase lasting 36 hours, and a treatment phase lasting 24 hours. During confinement, subjects abstained from smoking, which was confirmed by an expired carbon monoxide (CO) of 10 ppm or less as measured by a calibrated Bedfont Smokerlyzer (Bedfont Scientific Ltd, Harrietsham, United Kingdom). Prespecified CO measurements were obtained at baseline, immediately before randomization, and prior to dose administration. In addition, there were four random CO measurements during each treatment period. Subjects also abstained from alcohol and strenuous exercise 48 hours prior to each blood collection.

### Washout

The washout period began 24 hours after the study treatment was administered during the first treatment period and lasted from 5 to 7 days. Subjects were allowed to smoke cigarettes during the washout period, but reported to the study site 36 hours prior to drug administration for the second treatment period. Smoking abstinence was required during the second treatment period as it was during the first treatment period.

### Study Treatments

Subjects were randomly assigned to one of two dosing sequences in each study with the washout period between the different study treatments. The sequences included the test tablet followed by the Reference Product, or the Reference Product followed by the test tablet, respectively. Study treatments were administered as a single dose given at approximately 8:00 am, after a 10-hour overnight fast. Subjects placed the lozenge into their oral cavity and occasionally moved the lozenge from side to side to allow it to dissolve slowly, without chewing and while minimizing swallowing.

### Screening Visit

Subjects were recruited by the study site. All subjects participated in a screening visit at least 2 days before random assignment. During the screening visit, subjects underwent screening procedures that included collecting demographic information, medical history, smoking history, and a serum pregnancy test and contraception review if applicable. A physical and clinical exam was performed that included assessment of vital signs and a 12-lead electrocardiogram. Subjects level of nicotine dependency was measured using the Fagerstrom Test for Nicotine Dependence (FTND).

### Treatment Periods

The check-in visit occurred 2 days prior to the beginning of each treatment period to ensure that subjects abstained from nicotine use 36 hours prior to the first study treatment. During check-in, the inclusion and exclusion criteria were verified, and subjects underwent a brief physical exam, laboratory and urine testing, and serum pregnancy test and contraception review, if applicable.

The baseline visit occurred 1 day after the check-in visit. During the baseline visit, expired CO was measured. Subjects were randomized at the beginning of the first treatment visit, which occurred 36 hours before the study treatment was administered. Subjects underwent a brief physical exam with assessment of vital signs and prespecified and random CO measurements.

Subjects received a single dose of the study treatment at the beginning of the second treatment visit, which lasted for 24 hours. Blood sampling occurred pre-dose and post-dose.

At the end of the second treatment visit, subjects underwent a washout period of 5 to 7 days followed by the second treatment session in which subjects received their second treatment sequence.

### Sample Collection

Blood was collected for PK analyses during the first and second treatment periods of both studies. The blood sampling times were selected to capture nicotine absorption and elimination. The blood specimens were collected beginning 45 minutes prior to and up to and including 24 hours after study drug administration. Samples were collected at 45, 30, and 15 minutes predose, then 5, 10, 20, 30, 40, 50, 60, 75, and 90 minutes postdose, then 2, 3, 4, 6, 8, 10, 14, 20, and 24 hours postdose. For each time point, 6 mL of blood was collected via direct venipuncture into tubes containing K₂EDTA and plasma was harvested for further analyses. Plasma was split into two aliquots and stored frozen at −20° C. until analysis.

Plasma nicotine concentration was determined using a validated method of solid-phase extraction with ultra-high performance liquid chromatography and tandem mass spectrometry (LC-MS/MS) with a lower limit of quantitation for nicotine of 0.200 ng/mL

### Safety Evaluation.

The safety evaluation included documentation of all adverse events (AEs) that occurred during the study beginning at the time of signing the informed consent form to 5 days following the last treatment administration. An AE was defined as any untoward medical occurrence experienced by a study subject, regardless of whether it was considered related to the treatment drug. All treatment-emergent AEs (TEAEs) were summarized by primary SOC and PT and coded using MedDRA Version 21.0.

### Data Analyses

### Sample Size

The sample sizes calculations for both studies assumed a 20% dropout and nonevaluable rate. For the study of the 2 mg test tablet, at least 40 subjects were planned for screening to ensure that 32 subjects completed the study. This sample size would achieve 90% power at a 5% significance level, assuming the highest intrasubject coefficient of variation (CV) was 23%. The true ratio that was used in the sample size calculation was 1.05.

For the 4 mg test tablet, at least 37 subjects were planned for screening to ensure that 29 subjects completed the study. This sample size would achieve 90% power at a 5% significance level, assuming the highest intrasubject CV of 22%. The true ratio that was used in the sample size calculation was 1.05.

### Analysis Populations

There were three analysis populations. All patients who were randomly assigned, regardless whether they received study treatment or not, were part of the randomized population. The safety population included all subjects who received at least one dose of the study treatment. The PK population included all randomized subjects who completed both treatment periods and who had no major protocol deviations concerning PK.

### Statistical Analyses

There were two PK analysis sets (PKAS). PKAS1 included data from all subjects in the PK population. Subjects with a baseline nicotine concentration greater than 5% of the individual Cₘₐₓ in either study period were excluded. PKAS2 included only baseline-adjusted data from subjects in the PK population for which the relevant baseline-adjusted PK parameters (at least one of AUC or Cₘₐₓ) could be derived, including those with baseline nicotine concentrations greater than 5% of the individual Cₘₐₓ in either period.

The primary endpoint was to determine the bioequivalence of the test tablet to the Reference Product, which was assessed by pairwise comparison of the PK parameters (AUC₀₋ₜ, AUC_{0-∞}, and Cₘₐₓ) for the baseline-adjusted nicotine concentration profiles from PKAS1.

The PK parameters were calculated using Phoenix WinNonlin Version 7.0 or higher. A linear mixed-effects model was fit to the natural log (ln)-transformed PK variables (AUC₀₋ₜ, AUC_{0-∞}, and Cₘₐₓ) as the dependent variable, and treatment, period, and sequence as fixed effects. Subject nested within sequence was a random effect. Least-squares estimates of treatment effects were calculated and a 90% confidence interval (CI) for the treatment difference was computed. The treatment difference and its 90% CI were exponentiated to obtain the geometric mean ratios (GMR) between the test and reference products and its 90% CI. Bioequivalence was determined if 90% CIs of the GMRs of AUC₀₋ₜ, AUC_{0-∞}, and Cₘₐₓ fell completely within the 0.80-1.25 range.

The secondary endpoints included comparing PK parameters (T max, t 0.1/2, Kₑₗ), and safety of the test tablet and the Reference Product among subjects in PKAS1 and PKAS2. A nonparametric analysis for Tₘₐₓ was performed to compare treatment differences using the Wilcoxon Signed Rank test. Median difference (test-reference), the Hodges-Lehmann estimator, and estimated CI were used to examine the location shift in Tₘₐₓ (Hollander and Wolf, Nonparametric Statistical Methods, 2nd Edition. Hoboken, N.J.: Wiley; 1999). Tₘₐₓ was not in-transformed.

### RESULTS

### Subject Disposition and Demographics

### 2-mg Study

For the 2-mg study, 90 subjects were screened and 46 were included in the study. A total of 46 subjects were treated with the test tablet and 44 subjects were treated with the Reference Product. The safety population included all 46 subjects who were randomized. Six subjects were excluded from the PK population due to major protocol deviations including four subjects who missed a scheduled blood draw and two subjects who did not complete both treatment sessions. All 40 subjects in the PK population were included in the PKAS2 population. An additional 15 subjects were excluded from the PKAS1 population (n=25) due to protocol deviations, including six subjects who missed a scheduled blood draw, four subjects who did not complete both treatment periods, and five subjects whose predose plasma nicotine concentration was greater than 5% of Cₘₐₓ in at least 1 period.

Among the safety population, 65.2% of subjects were male and 34.8% were female. The majority of subjects were white at 71.2%, followed by 23.9% who were black, 2.2% who were Asian, and 2.2% who were American Indian or Alaska native. There were 4.3% of subjects who were of Hispanic or Latino ethnicity. The median age at baseline was 38.5 with a range of 19 to 54. The median body mass index (BMI) was 23.6 kg/m2 with a range of 19.2 to 27.2 kg/m2.

### 4-mg Study

For the 4-mg study, 61 subjects were screened and 37 were included in the study. A total of 36 subjects were treated with the test tablet and 35 subjects were treated with the Reference Product. The safety population included all 37 subjects who were randomized. There were three subjects who were excluded from the PK population because they did not complete both treatment periods. The PKAS2 population included all 34 subjects from the PK population. There were seven subjects who were excluded from the PKAS1 population (n=27) because their predose plasma nicotine concentration was greater than 5% of Cₘₐₓ in at least 1 period.

In the safety population, 67.6% of subjects were male and 32.4% were female. Similar to the 2-mg study, the majority of subjects were white (81.1%), followed by black (16.2%), or American Indian or Alaska native (2.7%). None of the subjects were of Hispanic or Latino ethnicity. The median age at baseline was 36 with a range of 20 to 55. The median BMI was 24.8 kg/m2 with a range of 20.4 to 26.7 kg/m2.

### Pharmacokinetics

### Primary Endpoints

The mean baseline-adjusted nicotine plasma profile of the 2 mg test tablet was similar to the 2 mg Reference Product, with a mean Cₘₐₓ of 4.613 ng/mL (standard deviation [SD], 1.390 ng/mL) and 5.146 ng/mL (SD, 1.652 ng/mL), respectively The mean Cₘₐₓ of the 4-mg test tablet was 8.779 ng/mL (SD, 3.205 ng/mL) compared to 8.972 (SD, 2.738 ng/mL) with the 4-mg Reference Product.

AUC₀₋ₜ was also similar between the test tablet and the Reference Product. In the 2-mg study, the baseline-adjusted mean AUC₀₋ₜ was 16.56 ng*hr/mL (SD, 6.973 ng*hr/mL) with the test tablet compared to 17.5 ng*hr/mL (SD, 7.878 ng*hr/mL) with the Reference Product. The geometric mean was 15.39 ng*hr/mL with a geometric CV of 39.4% with the 2-mg test tablet and was 16.19 ng*hr/mL with a geometric CV of 40.1% with the 2-mg Reference Product. In the 4-mg study, the mean AUC₀₋ₜ was 38.43 ng*hr/mL (SD, 18.58 ng*hr/mL) with the test tablet compared to 39.82 ng*hr/mL (SD, 18.83 ng*hr/mL) with the Reference Product. The geometric mean was 34.44 and 36.39 ng*hr/mL, with the test tablet and Reference Product, respectively, with a geometric CVs of 51.5% and 43.9%.

The test tablet demonstrated a similar baseline-adjusted AUC_{0-∞} as the Reference Product at both dose levels. The 2-mg test tablet had a mean AUC₀₋₂₈ of 17.80 ng*hr/mL (SD, 7.553 ng*hr/mL) compared to 18.79 ng*hr/mL (SD, 8.560 ng*hr/mL) with the Reference Product. The geometric mean was 16.53 and 17.38 ng*hr/mL with the test tablet and Reference Product, respectively, and the geometric CVs were 39.5% and 40.2%. The 4-mg test tablet resulted in a mean AUC_{0-∞} of 40.64 ng*hr/mL (SD, 19.57 ng*hr/mL) compared to 42.10 ng*hr/mL (SD, 19.53 ng*hr/mL) with the Reference Product, with geometric means of 36.39 and 38.54 ng*hr/mL, respectively. The geometric CV was 52.0% with the test tablet and 43.6% with the Reference Product. See FIG. 2 and Table 1 (FIG. 3).

Both the 2-mg and 4-mg test tablets were bioequivalent to their respective Reference Products. In the 2-mg study, the test tablet and the Reference Product were bioequivalent for AUC₀₋ₜ, AUC₀₋₂₈, and Cₘₐₓ. The geometric Least Square Mean (LSM) of the baseline-adjusted AUC₀₋ₜ was 15.87 ng*hr/mL with the test tablet compared to 16.64 ng*hr/mL with the Reference Product (GMR, 0.9538; 90% CI, 0.9060-1.0040; intrasubject CV, 10.19%). The geometric LSM of the AUC_{0-∞} was 17.06 ng*hr/mL and 18.08 ng*hr/mL with the test tablet and Reference Product, respectively (GMR, 0.947; 90% CI, 0.8946-0.9956; intrasubject CV, 10.14%). The Cₘₐₓ was also bioequivalent with a geometric LSM of 4.419 ng/mL with the test tablet and 4.950 ng/mL with the Reference Product (GMR, 0.8927; 90% CI, 0.8168-0.9757; intrasubject CV, 17.74%).

The 4-mg test tablet was bioequivalent to the 4-mg Reference Product for AUC₀₋ₜ, AUC_{0-∞}, or Cₘₐₓ. The geometric LSM of AUC_{0-∞} was 34.41 ng*hr/mL with the test tablet compared to 36.35 ng*hr/mL with the Reference Product (GMR, 0.9468; 90% CI, 0.8823-1.0160; intrasubject CV, 15.25%). The geometric LSM of AUC_{0-∞} was 36.32 ng*hr/mL and 38.50 ng*hr/mL with the test tablet and the Reference Product, respectively (GMR, 0.9434; 90% CI, 0.8782-1.0133; intrasubject CV 15.13%). The Cₘₐₓ was also bioequivalent between treatments, with a geometric LSM of 8.282 ng/mL with the test product and 8.527 ng/mL with the Reference Product (GMR, 0.9661; 90% CI, 0.8970-1.0406; intrasubject CV, 16.06%). See Table 2 (FIG. 4).

### Additional Secondary Endpoints

Tₘₐₓ was similar between the 2-mg treatments, which was reached at 1.056 hours (SD, 0.3872) with the 2-mg test tablet compared with 1.065 hours (SD, 0.3115) with the Reference Product.

As the 95% CI 3 median difference in nicotine Tₘₐₓ between the 2 mg test tablet and the Reference Product treatments contained 0, the Tₘₐₓ of nicotine was similar. The median of paired differences in Tₘₐₓ between the treatments was −0.094 with a median of −0.0225 (95% CI, −0.1785 to 0.1285; P=0.6662). The half-life of the 2-mg lozenges were similar, with a mean of 3.643 hr (SD, 3.307 hr) with the test tablet and 3.462 hr (SD, 3.130 hr) with the Reference Product.

Tₘₐₓ was also similar between the 4-mg treatments, which was reached in a mean of 1.423 hours (SD, 0.5538) with the 4-mg test tablet compared with 1.342 hours (SD, 0.4551) with the Reference Product. As the 95% CI of the median difference in nicotine Tₘₐₓ between the 4 mg test tablet and the Reference Product treatments contained 0, the Tₘₐₓ of nicotine was similar. The median of paired differences was 0.179 between the treatments with a median of 0.117 (95% CI, −0.1250 to 0.2535; P=0.2463).

The half-life was similar and occurred at a mean of 5.924 (SD, 3.2689) and 6.324 hours (SD, 3.8273) with the 4-mg test tablet and the Reference Product, respectively.

The Kₑₗ was also similar between the treatment groups in both studies. In the 2-mg study, the mean Kₑₗ was 0.276 l/hr (SD, 0.118 l/hr) with the test tablet compared to 0.2781 1/hr (SD, 0.111 l/hr) with the Reference Product. With the 4-mg test tablet, the mean Kₑₗ was 0.1610 l/hr (SD, 0.102 l/hr) compared with 0.154 l/hr (SD, 0.091 l/hr) with the Reference Product. See Table 3 (FIG. 5) and Table 4 (FIG. 6).

### Safety

The 2- and 4-mg test tablets were well-tolerated by study subjects, with no serious adverse events (SAEs), or deaths during either study among the safety populations. There were no discontinuations due to AEs in the 2-mg study, and one subject who received the commercially-available mini lozenge discontinued the study due to an AE unrelated to the study treatment.

In the 2-mg study, 50.0% of subjects experienced a treatment-emergent AE (TEAE), including 28.3% in the test tablet arm and 34.1% in the Reference Product arm. All of the AEs were mild in severity, except for a case of moderate pruritus in the mini lozenge arm.

The rate of suspected treatment-related TEAEs was also similar between arms, with 10.9% of subjects experiencing a treatment-related TEAE in the test tablet arm compared to 13.6% of subjects in the Reference Product arm. The most common treatment-related TEAEs were throat irritation, which occurred in three subjects in the test tablet arm and 2 subjects in the Reference Product am, and dyspepsia, which occurred in 2 subjects in the test tablet arm and 3 subjects in the Reference Product arm. There were no changes in the clinical laboratory or vital sign evaluations during the study.

In the 4-mg study, 43.2% of subjects developed a TEAE, including 36.1% of subjects with the test product and 17.1% of subjects with the Reference Product. All of the TEAEs were mild in severity. Suspected treatment-related TEAEs were higher with the test tablet, with 30.6% of subjects reporting a treatment-related TEAE compared to 11.4% with the Reference Product. Similar to the 2-mg study, the most common treatment-related TEAE was throat irritation, which occurred in five subjects during their use of the 4 mg test tablet and two subjects when using the 4 mg Reference Product. Other treatment-related TEAEs that occurred in at least 5% of subjects were hiccups (5.4%, 1 subject with each treatment), nausea (10.8%, 4 subjects with the prototype), oral discomfort (5.4%, 1 subject with each treatment), and dizziness (5.4%, 1 subject with each treatment). There were no changes in the clinical laboratory or vital sign evaluations during the study.

### DISCUSSION

The present studies demonstrated that the test tablet is bioequivalent to the Reference Product at both the 2 and 4 mg doses. The PK parameters of Cₘₐₓ, AUC₀₋ₜ, and AUC_{0-∞} were bioequivalent between the test tablet and the Reference Product. In addition, the secondary PK parameters, including Tₘₐₓ, t_{1/2}, and Kₑₗ, were also similar between the two.

The bioequivalence of the test tablet to the Reference Product was determined in two separate studies, one of the 2 mg dose and the other of the 4 mg dose. Comparisons cannot be made between the studies; however, the higher 4 mg dose resulted in a higher baseline-adjusted plasma nicotine concentration than the 2 mg dose. Both tablets were well-tolerated and there were no new safety signals observed. At both dosage levels, throat irritation was the most common TEAE reported by the subjects and was mild in severity. None of the subjects discontinued the study as a result of throat irritation or any other TEAE.

### CONCLUSIONS

The results of the studies demonstrate that the 2 and 4 mg test tablets are bioequivalent with the 2 and 4 mg Reference Products, respectively, with no significant difference in Cₘₐₓ, AUC₀₋ₜ, or AUC_{0-∞} or any of the secondary outcomes. The most common treatment-emergent adverse event was throat irritation, of which all cases were mild in severity. There were no serious adverse events.

Therefore, the test tablets provide smokers seeking to quit with a new oral NRT option to aid in smoking cessation and of tobacco dependence through the relief of nicotine withdrawal symptoms, including cravings.

### Abbreviations

ANOVA=analysis of variance;
AUC=area under the curve;
AUC₍₀₋ₜ₎, =area under the plasma concentration versus time curve at a certain time t) after drug administration;
AUC_{(0-infin)}=area under the plasma concentration versus time curve for total drug exposure;
CI=confidence interval;
Cₘₐₓ=maximal plasma nicotine concentration;
CV=coefficient of variation;
GMR=geometric mean ratio;
inf=infinity;
∞=infinity;
Kₑₗ=elimination rate constant;
LSM=least-squares mean;
SD=standard deviation.
t_{1/2}=half-life;
Tₘₐₓ=time to Cₘₐₓ

What is claimed is:

1. A tablet for intra-oral delivery of nicotine to a subject in need thereof which comprises:
(a) an orally disintegrating portion comprising a sensory marker/signal and a disintegration promoting excipient, and
(b) a troche/lozenge portion comprising a nicotine active agent, a buffer system, and a hydrophilic matrix.

2. A tablet according to claim 1 wherein the sensory marker/signal comprises an organoleptic stimulus.

3. A tablet according to claim 2 wherein the organoleptic stimulus comprises a flavor, and optionally a sweetener.

4. A tablet according to claim 3 wherein the flavor is mint.

5. A tablet according to claim 1 wherein the disintegration promoting excipient of (a) comprises a disintegration promoting excipient premix comprising mannitol, xylitol, dibasic calcium phosphate anhydrous, crospovidone, and microcrystalline cellulose.

6. A tablet according to claim 1 wherein the buffer system of (b) comprises sodium carbonate anhydrous and sodium bicarbonate in relative amounts by weight of about 10:1 to about 7:1; and the hydrophilic matrix comprises Hypromellose and xanthan gum in relative amounts by weight of about 0.75:1 to about 3:1.

7. A tablet according to claim 6 wherein (b) additionally comprises a lubricant which is magnesium stearate in an amount of at least about 2 wt. % but no more than 3 wt. %,

8. A tablet according to claim 1 wherein (a) is capable of disintegrating in the oral cavity in about 60 seconds or less.

9. A tablet according to claim 1 wherein (b) disintegrates or dissolves in the oral cavity over a period of from about 5 to about 20 minutes.

10. A tablet according to claim 1 wherein the nicotine active agent of (b) comprises Nicotine Polacrilex.

11. A tablet according to claim 6 wherein the Nicotine Polacrilex is present in an amount equivalent to 2 mg of nicotine.

12. A tablet according to claim 6 wherein the Nicotine Polacrilex is present in an amount equivalent to 4 mg of nicotine.

13. A tablet according to claim 1 comprising the nicotine active agent in an amount of 2 mg or 4 mg (calculated as nicotinee), wherein said tablet is bioequivalent to the corresponding 2 mg or 4 mg Reference Product, and bioequivalence is established by at least one pharmacokinetic parameter that is selected from (i) a confidence interval for mean AUC(₀₋ₜ) between about 80% and about 125%; (ii) a confidence interval for mean AUC (_{0-infin}) between about 80% and about 125%; (iii) a confidence interval for mean Cₘₐₓ between about 80% and about 125%; (iv) a confidence interval for mean Tₘₐₓ between about 80% and about 125%; and (v) combinations of any of (i)-(iv).

14. A tablet according to claim 1 comprising the nicotine active agent in an amount of 2 mg or 4 mg (calculated as nicotine base), wherein said tablet is bioequivalent to the corresponding 2 mg or 4 mg Reference Product, and bioequivalence is established by at least one pharmacokinetic parameter that is selected from (i) a confidence interval for mean AUC(₀₋ₜ) between about 70% and about 143%; (ii) a confidence interval for mean AUC (_{0-infin}), between about 70% and about 143%; (iii) a confidence interval for mean Cₘₐₓ between about 70% and about 143%; (iv) a confidence interval for mean Tₘₐₓ between about 70% and about 143%; and (v) combinations of any of (i)-(iv).

15. A tablet according to claim 13 wherein bioequivalence is established by pharmacokinetic parameters (i), (ii) and (iii).

16. A tablet according to claim 14 wherein bioequivalence is established by pharmacokinetic parameters (i), (ii) and (iii).

17. A method for reducing withdrawal symptoms associated with smoking or use of a tobacco containing material; and/or for obtaining a reduction of the urge or craving to smoke or use tobacco containing material; and/or for providing a sense of smoking satisfaction without smoking, comprising the steps of replacing at least partly the tobacco containing material with a tablet of the invention, administering the tablet into the oral cavity of the subject and allowing the nicotine of the tablet to be released in the saliva in the oral cavity and absorbed by the subject into the systemic circulation of the subject.

18. A direct compression process for preparing a tablet according to claim 1 comprising:
(i) separately blending and optionally sieving the ingredients comprising each of (a) and (b), in the absence of granulation;
(ii) optionally pre-compressing each of (a) and (b); and
(iii) compressing (a) and (b) together to form the tablet.
